(19) United States
(12) Patent Application Publication
(10) Pub. No.: US 2005/0277881 A1
(43) Pub. Date: Dec. 15, 2005

(54) Safety needles with locking sheaths

(76) Inventor: Wilmer L. Sibbitt JR., Albuquerque, NM (US)

Correspondence Address: ROBERTS ABOKHAIR & MARDULA, SUITE 1000, 11800 SUNRISE VALLEY DRIVE, RESTON, VA, 20191, US

(21) Appl. No.: 11/150,381

(22) Filed: Jun. 10, 2005

Related U.S. Application Data
(60) Provisional application No. 60/578,998, filed on Jun. 12, 2004.

(51) Int. Cl. A61M 5/00

(52) U.S. Cl. 604/110; 604/263

(57) ABSTRACT

A cap or sheath locks onto the needle or other sharp, thus permanently inactivating the needle or other sharp, preventing any further use of the device, and permanently isolating the contaminated sharp. Four components are a needle or sharp to accommodate a locking collar, a sheath with a external, internal, or horizontal track to accommodate a locking collar, a locking collar that moves inferiorly within or outside the sheath, or horizontally that binds to the sheath or needle, permanently locking the needle in the sheath and mechanically preventing the needle from moving from the sheath, and a locking mechanisms for these devices. Handles are advantageously provided for the sheath to permit safe recapping or resheathing so that the needle can be bound in the sheath and inactivated.

### CROSS REFERENCE TO RELATED APPLICATIONS

This application claims priority benefit under 35 U.S.C. § 119(e) of provisional application No. 60/578,998, filed Jun. 12, 2004. The 60/578,998 application is incorporated by reference herein, in its entirety, for all purposes.

### FIELD OF THE INVENTION

The present invention relates generally to the field of sharp medical devices. More specifically, the present invention relates to the placement of protective covers of the sharp portions of a medical device so as to prevent inadvertent sticks.

### BACKGROUND OF THE INVENTION

Accidental penetration of the skin from sharp instruments is one of the most common modes of transmission of fatal or debilitating infectious diseases to health care workers. Hepatitis B, hepatitis C, and HIV (the AIDS virus) in the health care environment are typically transmitted from needle sticks and result in years of debilitating illness, loss of productivity, worker's compensation payments, medical expenses, and accelerated mortality. Health care workers most susceptible to needle sticks include nurses and laboratory workers, but physicians, dentists, dialysis workers, oral surgeons, medical waste workers, and animal handlers are also exposed.

Using a standard cap, recapping a hypodermic needle is an extremely dangerous procedure, entailing a 6% risk of needle stick of per capping attempt. OSHA requires regular instruction of health care workers in techniques to prevent accidental needle sticks, yet needle sticks, especially those from recapping, remain an important mechanism for transmission of virulent infectious agents to health care workers. Any advances in medical instrument design that would limit or prevent needle sticks would markedly reduce the health risks from infectious diseases for health care workers and would result in considerable savings from lost productivity, medical costs, litigation, and compensation payments. Most importantly, the health and safety of health care workers would be improved.

Hypodermic needles have been used for many years in industry, research, and medical practice. These needles, which consist of a hollow metal tube sharpened on one end in order to penetrate the skin or other substance, are attached to a syringe which is used to aspirate or inject volumes of fluid through the hollow barrel of the needle. In medical practice, hypodermic needles are most often used to inject medications into skin, subcutaneous tissues, muscle, blood vessels, or other tissues. Hypodermic needles are also used to aspirate fluid from body cavities and to transfer medications, samples, or reagents from stoppered bottles or other containers to other containers or devices.

Before use, the hypodermic needle is sharp but sterile. However, after use, the needle becomes contaminated with blood, body fluids, or residual fluid remaining from aspiration of samples from bottles. These contaminated hypodermic needles remain very sharp and easily penetrate the skin, directly depositing infectious agents into the body tissues of the medical worker. These injuries have a high incidence of transmission of hepatitis B and hepatitis C, as well as other infectious agents. Needle sticks from hypodermic needles occur in several ways: 1) accidental sticks that occur from improperly discarded uncapped needles or capped needles that become uncapped in refuse or by impact, 2) accidental sticks that occur from the act of improperly discarding needles, 3) sticks that occur from attempts to recap the contaminated needle, 4) sticks that occur to the operator from an uncapped needle not in immediate use, but still on the equipment tray or in the operating field, 5) sticks that occur in the operating field from a misdirected needle, or 6) intentional sticks.

One approach to this problem has been the use of syringes with retractable needles, where as the syringe and the needle are inactivated by retracting the needle within the syringe. Although this type of retractable needle is acceptable for a simple maneuver where a medication is aspirated into a syringe from a vial then injected into a patient or medical solution, there are serious disadvantages. First, the needle becomes dull after pushing through a stopper, and then causes increased pain for the patient because of the dull needle tugs and stimulates the nerves of the skin. Moreover, certain needle designs core the stopper, resulting in a piece of rubber being injected into the patient. Also, medication or bacteria may contaminate the outside of the needle barrel, causing misadministration of medication into the skin or subcutaneous tissues or the appearance of an infection.

Because of the above problems, medical workers like to change to a fresh needle before injecting a medication. Most syringes with retractable needles do not permit this. Another problem with syringes with retractable needles is that they need to be recapped if the medication is not going to be immediately administered. Thus, there is danger of a needle stick on recapping. Finally, for medical procedures, different sizes of needles need to be used for different components of the procedure, thus, needles are often changed on syringes. Furthermore, aspirated fluid often needs to be kept in a syringe for analysis, but the needle cannot be inactivated in most retractable syringes without isolating the aspirated fluid.

Safety needles have also been proposed, where the needle has an attached sheath that is slid over or otherwise covers the needle after use. Although these types of needles may permit needles to be inactivated and changed between different components of a medical procedure and obviates recapping to inactivate, often they have to be recapped anyway for transport before use. Another major problem with these types of safety needles are that the safety mechanism increases the physical profile of the needle, resulting in a physical obstruction during a medical procedure and thus decreased line of sight and on occasion, an actually physical obstruction of the safety device interfering with the medical procedure. In addition, many of these safety needles, depending on the design, will fit on only one size of syringe so many duplicative needles are required for different syringe sizes.

Because of these features, nurses, physicians, and other medical workers generally do not like syringes with retractable needles and similarly are dissatisfied with most safety needles for administering medications or performing syringe procedures.

What is needed is both a mechanism to safety recap needles and sharp, to changes needles as desired and appropriate, to provide a low profile needle that permits exact control by the operator, and to provide a locking sheath that permanently inactivate a needle it so that needle sticks cannot occur and contaminated or noxious contents are isolated within the needle.

### SUMMARY OF THE INVENTION

The present invention overcomes all the above-noted difficulties of the other approaches to this problem. The present invention has of a cap or sheath which locks onto the needle or other sharp, thus permanently inactivating the needle or other sharp, preventing any further use of the device, and permanently isolating the contaminated sharp.

One aspect of the invention is a needle or sharp to accommodating a locking collar or collar equivalent.

Another aspect of the invention is a sheath with a track (external, internal, or horizontal) to accommodate a locking collar or collar equivalent.

Yet another aspect of the invention is a locking collar or equivalent that moves inferiorly within or outside the sheath, or horizontally that binds to the sheath or needle, permanently locking the needle or sharp in the sheath and mechanically preventing the needle from moving from the sheath.

Still another aspect of the invention is a locking mechanisms for such devices.

Another aspect of the invention is to provide one or more handles for the sheath to permit safe recapping or re-sheathing so that the needle can be bound in the sheath and inactivated.

### BRIEF DESCRIPTION OF THE FIGURES

FIG. 1 illustrates a needle with a protective sheath according to one embodiment of the present invention, with a locking collar in a utility position.

FIG. 2 illustrates a needle with a protective sheath according to the embodiment of FIG. 1, with the locking collar in a fully locked position.

FIG. 3 illustrates a needle according to the embodiment of FIG. 1.

FIG. 4 illustrates a protective sheath according to the embodiment of FIG. 1.

FIG. 5 illustrates a front elevation view of a locking collar according to the embodiment of FIG. 1.

FIG. 6 illustrates a side elevation view of a locking collar according to the embodiment of FIG. 1.

FIG. 7 illustrates a bottom plan view of a locking collar according to the embodiment of FIG. 1.

FIG. 8 illustrates a sectional view of a locking collar according to the embodiment of FIG. 1.

FIG. 9 illustrates a front elevation view of a locking collar according to an alternate embodiment.

FIG. 10 illustrates a side elevation view of a locking collar according to the alternate embodiment of FIG. 9.

FIG. 11 illustrates a bottom plan view of a locking collar according to the alternate embodiment of FIG. 9.

FIG. 12 illustrates a sectional view of a locking collar according to the alternate embodiment of FIG. 9.

FIG. 13 illustrates a needle with a protective sheath according to another embodiment of the present invention, with an internal locking collar in a utility position.

FIG. 14 illustrates a needle with a protective sheath according to the embodiment of FIG. 13, with the internal locking collar in a fully locked position.

FIG. 15 illustrates an elevation view of the needle according to the embodiment of FIG. 13.

FIG. 16 illustrates an elevation view of the protective sheath according to the embodiment of FIG. 13.

FIG. 17 illustrates an elevation view of the internal locking collar according to the embodiment of FIG. 13.

FIG. 18 illustrates a section view of the internal locking collar according to the embodiment of FIG. 13.

FIG. 19 illustrates a bottom plan view of the internal locking collar according to the embodiment of FIG. 13.

FIG. 20 illustrates a needle with a protective sheath according to yet another embodiment of the present invention, with a lateral locking collar in a utility position.

FIG. 21 illustrates a needle with a protective sheath according to the embodiment of FIG. 20, with the lateral locking collar in a fully locked position.

FIG. 22 illustrates an elevation view of the needle according to the embodiment of FIG. 20.

FIG. 23 illustrates a side elevation view of the protective sheath according to the embodiment of FIG. 20.

FIG. 24 illustrates a front elevation view of the protective sheath according to the embodiment of FIG. 20.

FIG. 25 illustrates an elevation view of the lateral locking collar according to the embodiment of FIG. 20.

FIG. 26 illustrates a plan view of the lateral locking collar according to the embodiment of FIG. 20.

FIG. 27 illustrates a plan view of the lateral locking collar according to an alternate embodiment of FIG. 20.

FIG. 28 illustrates an embodiment of the invention similar to that shown in FIG. 1, including the additional feature of a handle.

FIG. 29 illustrates an embodiment of the invention similar to that shown in FIG. 13, including the additional feature of a handle.

FIG. 30 illustrates an embodiment of the invention similar to that shown in FIG. 20, including the additional feature of a handle.

FIG. 31 illustrates a detail view of the protective cap similar to that shown in the embodiment of FIG. 29, showing dimensioning of the handle.

FIG. 32 illustrates a detail view of the protective cap similar to that shown in the embodiment of FIG. 29, showing an alternate handle shape.

FIG. 33 illustrates a detail view of the protective cap similar to that shown in the embodiment of FIG. 29, showing an additional handle connected to the internal locking collar.

### DETAILED DESCRIPTION

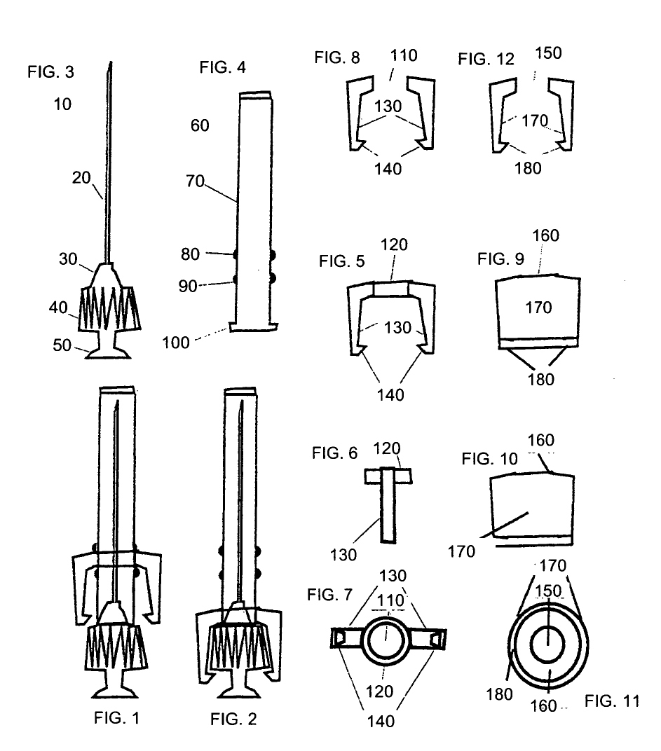

FIGS. 1-12 illustrate a safety needle with an external locking mechanism.

Referring to FIG. 1, a needle with a protective sheath according to one embodiment of the present invention is illustrated, with a locking collar in a utility position. This embodiment has a safety needle with an external locking collar disposed outside of the sheath.

Referring to FIG. 2, a needle with a protective sheath according to the embodiment of FIG. 1 is illustrated, with the locking collar in a fully locked position. Locking fittings on the end of the collar interdigitate and lock into the corresponding fittings on the needle neck.

Referring to FIG. 3, a needle according to the embodiment of FIG. 1 is illustrated. The needle 10 has a metal barrel 20, a hub 30, a neck 40, and a fitting 50, such as a Luer. The enlarged neck 40 can function as a grip and can be textured as shown, or can not be a grip, and can be non-textured and smaller than shown, but generally will be equal or greater in diameter to the outside diameter of the cap or sheath. The outside surface of the neck can be parallel to the axis of the metal barrel of the needle, or can have an angle to the surface (as shown) to facilitate movement of the locking device over the neck. The locking surface on the neck can be anywhere on the neck. However, the locking surface on the neck typically could be the fitting (inferior) side as shown, on the grip side (lateral side), or on the surface adjoining the hub (superior side), with the only requirement that there be a fitting to interdigitate and receive the corresponding fitting of the locking collar. In the case of a screw fitting, this surface would include a-threaded fitting which would interdigitate with a corresponding fitting on the collar. This could be reversible, locking or “child-proof” threads.

Referring to FIG. 4, a protective sheath according to the embodiment of FIG. 1 is illustrated. The hollow cap or sheath 60 is usually a cylinder composed of plastic which is closed at one end and open at the other end to accommodate the needle. This cap can be a wrench cap (with fittings that mesh with the needle hub used to seat the needle fitting into a medical device) or a non-wrench cap (used to cover the device only), The sides 70 of the sheath 60 can be parallel to the needle barrel as shown, or can be angled in any direction. In a number of situations it will be valuable to have stops 80, 90 to limit careless or accidental movement of the locking collar on the sheath, particularly when the needle is being seated on a medical device, being uncapped or recapped, or otherwise being moved about or manipulated. These stops 80, 90 can be permanent or can be overcome with force, and thus can be on the same side of the collar when locked (as shown in FIG. 2) or can be on opposite sides of the collar when locked. A permanent stop 100 is used to mechanically bind the collar to both the sheath 60 and the needle 10 when in the locked position.

According to alternate configurations (not shown), a stop 90 could function as the permanent stop. With or without the stops, the collar could also be limited in motion by having a metal or plastic spring, or compressible foam which would be placed on the cap 60, would be limited in its motion by the permanent stop 100, and then the collar would be placed on top spring so that the spring would reside between the locking collar and the permanent stop 100. This would be another method other than the stop 90 to prevent unwanted movement of the locking collar down upon the neck of the needle.

Two examples are shown of locking collars that slide in a longitudinal direction on the cap or sheath, locking the needle into the cap. FIGS. 5-8 illustrate an example with two or more tines or arms that lock the cap or sheath onto the needle neck. FIGS. 9-12 illustrate an example with a locking collar that is a continuous cylindrical piece without arms or tines.

Referring to FIG. 5, a front elevation view of a locking collar according to the embodiment of FIG. 1 is illustrated. The locking collar has a circular or band-like fitting 120 that is generally continuous which creates the aperture or hole that accommodates the cap or sheath. Tines or arms 130, which are attached to the circular fitting 120, have a locking fitting 140.

Referring to FIG. 6, a side elevation view of a locking collar according to the embodiment of FIG. 1 is illustrated. The circular fitting 120 and a tine 130 are shown.

Referring to FIG. 7, a bottom plan view of a locking collar according to the embodiment of FIG. 1 is illustrated. A circular or band-like fitting 120, which is generally continuous, creates the aperture or hole 110 that accommodates the cap or sheath. Tines or arms 130 are attached to the circular fitting 120. Locking fittings 140 on the ends of the tines 130 interdigitate with and lock into the corresponding fittings on the needle neck.

Referring to FIG. 8, a sectional view of a locking collar according to the embodiment of FIG. 1 is illustrated. An aperture or hole 110 that accommodates the cap or sheath and is formed by a circular or band-like fitting which is generally continuous. This fitting need not be circular however, and could accommodate other geometric shapes (rectangular, grooves, ovals, etc) as long as they correspond to the external surface of sheath or cap so that the collar can move in a longitudinal direction (long dimension of the cap). Tines or arms 130 are attached to the circular fitting that have a locking fitting 140 that locks into the corresponding fitting of the needle neck. In this case, engagement of the locking fitting 140 is on the fitting side (inferior) of neck, but could on the grip side (lateral), or the hub side (superior). Two tines 130 are illustrated, but a larger number is also possible.

Referring to FIG. 9, a front elevation view of a locking collar according to an alternate embodiment is illustrated. An aperture or hole 160 is sized to accommodate the cap or sheath. The external sides 170 of the continuous circular fitting and these sides that have a locking fitting 180 on the end. These locking fitting s 180 are internal if the collar is locking to the base of the neck and sides of the needle neck, but could be internal or external if they lock into the top of the needle neck.

Referring to FIG. 10, a side elevation view of a locking collar according to the alternate embodiment of FIG. 9 is illustrated. The circular hole 160 accommodates the cap. The sides 170 of the collar are shown.

Referring to FIG. 11, a bottom plan view of a locking collar according to the alternate embodiment of FIG. 9 is illustrated. The roof 160 of the collar is generally continuous so as to create the aperture or hole 150 that accommodates the cap or sheath. The external side surfaces 170 of the collar are connected at the bottom with locking fittings 180.

Referring to FIG. 12, a sectional view of a locking collar according to the alternate embodiment of FIG. 9 is illustrated. An aperture or hole 150 that accommodates the cap or sheath and is formed by the roof of the fitting and is generally continuous. This fitting need not be circular however, and could accommodate other geometric shapes (rectangular, grooves, ovals, etc) as long as they correspond to the external surface of sheath or cap so that the collar can move in a longitudinal direction (long dimension of the cap). The internal sides 170 of the continuous locking collar terminate at the end of the cylinder as a locking fitting 180 that locks into the corresponding fitting of the needle neck. In the illustrated case the needle neck is on the fitting side of the neck, but could on the grip side (lateral), or the hub side (superior). This locking fitting 180 can be continuous or intermittent, but if intermittent at least 2 are needed for a very firm locking device. Although one could also suffice, such a configuration would not provide optimal locking stability.

It is anticipated that the external locking collar, which in the in the above discussion uses the cap or sheath itself for a track to control the motion of the collar, could instead use linear longitudinal tracks on sheath which would interdigitate with corresponding components on the collar or collar equivalent. Also, in the present instance it is shown that the male component or the locking mechanism is on the collar which fits over the female component of the locking mechanism on the needle base, hub, or neck. However, this could be reversed, where the male component would instead be on the needle base, hub, or neck and would lock over the female component of the collar or collar equivalent.

Examples of locking devices that would function with these designs includes tapered and/or interlocking rings or tabs; oppositely directed and interlocking dentates or projections whether abrupt or tapered; interlocking rings, ridges, or shaped projections that are trapped in a space created by a tapered dentate or projection and a mating surface that accommodates this projection, or oppositely directed dentates or projections; locking tines with or without male and female components, screw and threaded fittings including standard, incomplete, and “child proof.” These are only examples, and many other locking mechanisms are possible and anticipated between the outer collar or collar equivalent and the base, hub, or neck of the needle.

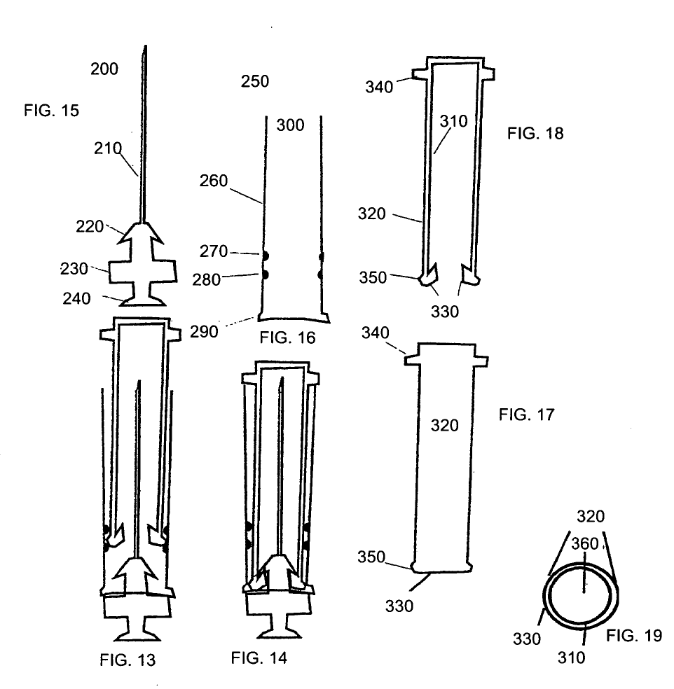

Referring to FIGS. 13-19, a safety needle is illustrated with an internal locking collar inside the sheath or cap.

Referring to FIG. 13, a needle with a protective sheath according to another embodiment of the present invention is illustrated, with an internal locking collar in a position that permits the needle to be uncapped and used.

Referring to FIG. 14, a needle with a protective sheath according to the embodiment of FIG. 13 is illustrated, with the internal locking collar in a fully locked position.

Referring to FIG. 15, an elevation view of the needle according to the embodiment of FIG. 13 is illustrated. The needle 200 has a metal barrel 210, a hub 220, a neck 230, and a fitting 240, such as a Luer. The neck 230 can function as a grip and can be textured, or can not be a grip, and can be non-textured and smaller than shown, but generally will contact the outside cap or sheath. The outside surface of the neck can be parallel to the axis of the metal barrel of the needle, or can have an angle or fittings so that the cap can be used as a wrench. The locking surface can be on the hub or neck here it is shown on the hub 220. On the hub 220, the locking surface can be can be anywhere, however, the locking surface on the neck on the surface adjoining the hub (superior side) with the only requirement that there be a fitting to interdigitate and receive the corresponding fitting of the locking collar. In the case of a screw fitting, this surface would include a threaded fitting which would interdigitate with a corresponding fitting on the collar. This could be reversible, locking or “child-proof” threads.

Referring to FIG. 16, an elevation view of the protective sheath according to the embodiment of FIG. 13 is illustrated. The hollow cap or sheath 250 is usually a cylinder composed of plastic which is open at both ends to accommodate internally both the needle and the locking collar. This cap can be a wrench cap (with fittings that mesh with the needle hub used to seat the needle fitting into a medical device) or a non-wrench cap (used to cover the device only). The opening on the superior end 300 accommodates the internal locking collar. The sides 260 of the sheath can be parallel to the needle barrel as shown, or can be angled in any direction. In a number of situations it will be valuable to have stops 270, 280 to limit careless or accidental movement of the locking collar within the sheath or cap, particularly when the needle is being seated on a medical device, being uncapped or recapped, or otherwise being moved about or manipulated. These stops 270, 280 can be permanent or can be overcome with force (as shown), and thus can be on the same side of the collar when locked (as shown) or can be on opposite sides of the collar when locked. Finally, the inferior surface of the cap or sheath 290 is necessary to mechanically bind the collar to both the sheath and the needle when in the locked position. With certain designs a stop 280 could function as the permanent stop. With or without the stops, the collar could also be limited in motion by having a metal or plastic spring, or compressible foam which would be placed within the cap 250, would be limited in its motion by the stops 270, 280, 290, and then the collar would be placed either on top of the spring or within the spring so that the spring would reside between the locking collar and the stops 270, 280, 290. This would be another method other than the stop 280 to prevent unwanted movement of the locking collar down upon the needle.

FIGS. 17 and 18 illustrate an example of an internal locking collar which would slide in a longitudinal direction within the cap or sheath, and bind to the needle hub or neck locking the needle into the cap. This exemplary embodiment has a locking collar that is a continuous cylindrical piece, although other shapes including an incomplete cylinder with arms or tines and locking mechanisms are contemplated.

Referring to FIG. 17, an elevation view of the internal locking collar according to the embodiment of FIG. 13 is illustrated. A stop 340 is disposed on the external sides 320 of the internal locking collar and these sides 320 have a locking fitting 330 on the end. These are internal if the collar is locking to the base of the neck and sides of the needle neck, but could be internal or external if they lock into the top of the needle neck. An inferior stop 350 is configured to mesh with the stops 270, 280 of the collar.

Referring to FIG. 18, a section view of the internal locking collar according to the embodiment of FIG. 13 is illustrated. An inner surface 310 of the collar is shown. This fitting need not be circular however, and could accommodate other geometric shapes (rectangular, grooves, ovals, etc) as long as they correspond to the external surface of sheath or cap so that the collar can move in a longitudinal direction (long dimension of the cap). The external sides 320 of the internal locking collar and at the end of the cylinder is a locking fitting 330 that locks into the corresponding fitting of the needle hub or neck, which in this case is on the hub 220. These locking fittings can be continuous or intermittent, but if intermittent at least 2 are useful to provide a very firm locking device. Although one could also suffice, is not optimal for locking stability. A stop 340 mechanically binds the inner locking collar to the cap or sheath. This could abut the end of the cap as shown, or could interdigitate with an internal stop in the cap (not shown). An inferior stop 350 is configured to mesh with the stops 270, 280 of the collar 250.

Referring to FIG. 19, a bottom plan view of the internal locking collar according to the embodiment of FIG. 13 is illustrated. The internal surface of the collar with the locking mechanisms 310 is shown. The roof 360 of the collar is generally continuous and also functions as the roof of the cap or sheath. The external side 320 surfaces of the collar come down to the end 330 of the collar that presents the locking fittings 310 on the end of the collar which interdigitate and lock into he corresponding fittings on the needle neck.

As in the first embodiment, the internal external locking collar, which in the above examples uses the cap or sheath itself for a tract to control the motion of the collar, could instead use linear longitudinal tracks within the sheath which would interdigitate with corresponding components on the collar or collar equivalent. Also, in the present instance it is shown that the male component or the locking mechanism is on the collar which fits over the female component of the locking mechanism on the needle base, hub, or neck; however, this could be reversed, where the male component would instead be on the needle base, hub, or neck and would lock over the female component of the collar or collar equivalent

Also, as in the first embodiment, examples of locking devices that would function with these designs includes tapered and/or interlocking rings or tabs; oppositely directed and interlocking dentates or projections whether abrupt or tapered; interlocking rings, ridges, or shaped projections that are crapped in a space created by a tapered dentate or projection and a mating surface that accommodates this projection, or oppositely directed dentates or projections; locking tines with or without male and female components, screw and threaded fittings including standard, incomplete, and “child proof.” These are only examples, and many other locking mechanisms are possible and anticipated between the inner collar or collar equivalent and the base, hub, or neck of the needle.

Although the previous two described embodiments effectively lock the cap or sheath to the needle or other sharp device, they are limited by the fact that to lock the cap to the needle, the needle must be seated in a rigid medical device or the needle hub or butt placed on a rigid surface so that the force placed on the collar causes locking rather than pushing the needle out of the cap or sheath. If collar is depressed rapidly while the needle is with the sheath, but not seated in a rigid device or placed against a hard surface, the needle may actually be dangerously ejected. Thus, there are limitation to these types of safety needles.

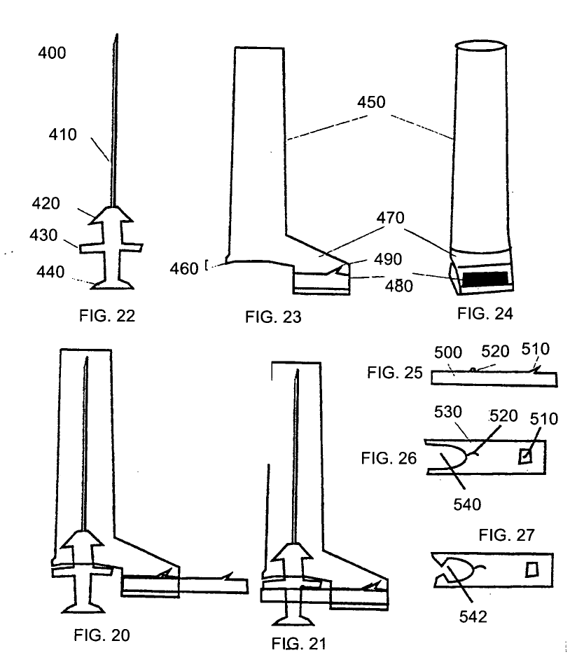

What is needed is a locking collar that locks the needle within the sheath or cap while the needle is seated on a device or when the needle is free, where putting against a hard surface is not necessary. A safety needle with a lateral locking collar accomplishes all of these goals. FIGS. 20-27 illustrate such an embodiment of the present invention.

Referring to FIG. 20, a needle with a protective sheath according to yet another embodiment of the present invention is illustrated, with a lateral locking collar in a position that permits uncapping and use of the needle.

Referring to FIG. 21, a needle with a protective sheath according to the embodiment of FIG. 20 is illustrated, with the lateral locking collar in a fully locked position.

Referring to FIG. 22, an elevation view of the needle according to the embodiment of FIG. 20 is illustrated. The needle 400 has a metal barrel 410, a hub 420, a neck 430, and a fitting 440, such as a Luer fitting. The neck 430 can function as a grip and can be textured, or can not be a grip, and can be non-textured and smaller than shown, but generally will contact the outside cap or sheath. The outside surface of the neck can be parallel to the axis of the metal barrel of the needle, or can have an angle or fittings so that the cap can be used as a wrench. The locking surface can be on the hub 420 or neck 430 and can be a single area of different diameter or multiple areas, creating a “notch” for a locking device. The locking device can be on the needle hub or the cap or both. Here it is shown on the cap 490. The hub or neck are important for providing a surface and plane for the needle to be locked into the cap or sheath.

Referring to FIG. 23, a side elevation view of the protective sheath according to the embodiment of FIG. 20 is illustrated. Referring to FIG. 24, a front elevation view of the protective sheath according to the embodiment of FIG. 20 is illustrated. The hollow cap or sheath is usually a cylinder composed of plastic which is open at both ends to accommodate internally both the needle and the locking collar. Here is shown in a lateral section and a partial frontal view. This cap can be a wrench cap (with fittings that mesh with the needle hub used to seat the needle fitting into a medical device) or a non-wrench cap (used to cover the device only}. The sides of the sheath 450 can be parallel to the needle barrel as shown, or can be angled in any direction. The surface 460 that contacts the needle hub or neck and this contact point can be external or internal to the cap and sheath, and can have interdigitating features so that it acts as a “wrench” on the needle hub or neck. An extension 470 of the cap that holds a track 480 so that the lateral locking collar can move in this track. Here the track 480 is shown to be internal, but this could be made on the external surface with appropriate grooved joints that permit linear motion of the collar in the track. This track 480 is shown on the extension 470 of the cap, but the track could be in the cap itself, and could use the hub 420 rather than the neck 430 to lock the needle into the cap. Also, to prevent careless locking of the cap, a guard could protrude from the extension 470 either superior or inferior to limit careless or accidental movement of the locking collar within the sheath or cap, particularly when the needle is being seated on a medical device, being uncapped or recapped, or otherwise being moved about or manipulated. Also a temporary or permanent stop 490 can be integrated in the track 480 which would interdigitate with a temporary stop on the locking collar.

These stops are can be permanent or can be overcome with force, and thus can be on the same side of the collar when locked (as shown) or can be on opposite sides of the collar when locked. With certain designs the stop 490 could function as this permanent stop. With or without the stops, the collar could also be limited in motion by having a metal or plastic spring, or compressible foam which would be placed within or outside the track 480, would be limited in its motion by the stops, and then the collar would be placed either on top of the spring or within the spring so that the spring would reside between the locking collar and the stops. This would be another method other than the stop 490 to prevent unwanted movement of the locking collar down upon the needle.

FIGS. 25-27 show examples of an lateral locking collar which would slide in a lateral (horizontal) direction into the cap or sheath, and bind to the needle hub or neck locking the needle into the cap. These figures illustrate the example of a lateral locking collar that is a continuous generally rectangular piece with a half circle as the portion that interdigitates with the needle, although other shapes including a full circle, arms or tines or other projections and locking mechanisms are anticipated and claimed.

Referring to FIG. 25, an elevation view of the lateral locking collar according to the embodiment of FIG. 20 is illustrated. The body 500 of the collar is shown. In this case, the body 500 is generally has a smooth or planar surface to move with the track 480; however, the surface in contact with the track 480 need not be strictly planar and could have a complex surface consisting of curved grooves which mates with corresponding grooves on the cap or sheath or on the cap extensions 470 providing a track to move in a linear lateral direction, providing the same mating function. These tracks could be internal, or external on the sides, top, or bottom of the sheath. The body 500 can have other geometric shapes (rectangular, columnar, etc) as long as these shapes correspond shape of the track 480 of sheath or cap so that the collar can move in a lateral (horizontal) direction (short dimension of the cap). The permanent locking mechanism of the lateral locking collar, here consists of a dentate 510 which locks into a corresponding receptacle 490 in the track 480. However, many other locking mechanisms besides dentates-in-hole (opposing dentates, ridge-in-furrow, peg-in-hole, slot and notch, and others) are possible and anticipated. The locking mechanism need not be in the tract, but could also be on the needle hub or neck, or could be on the opposing side of the cap or sheath. These locking fittings can be continuous or intermittent, although one, as shown, could also suffice. A temporary stop 520 is shown that temporarily restricts the motion of the locking collar so that it does not move unintentionally during manipulations. This temporary stop can be overcome with force.

Referring to FIG. 26, a plan view of the lateral locking collar according to the embodiment of FIG. 20 is illustrated. The superior surface 530 of the lateral locking collar is shown. The permanent or temporary locking device 510, a dentate in this case. The temporary stop 520 is shown. The half parabolic or oval cutaway 540 is the surface of the lateral locking collar that mates with the needle neck or hub, and restricts the motion of the needle by providing a mechanical obstruction to neck 430 or hub 420 by binding its inferior surface, so the needle or sharp cannot be removed from the cap or sheath. As mentioned the surfaces 430, 420 or other surfaces on the needle base, could have corresponding locking surfaces to the locking collar. This cutaway section 540 need not be “U-shaped” as shown, but have other shapes (open rectangular, notched (being the locking surface itself), or even circular, elliptical, or eccentric, where it could be a moveable part of the cap or sheath).

Referring to FIG. 27, a plan view of the lateral locking collar according to an alternate embodiment of FIG. 20 is illustrated. This is accomplished by having the binding surface of the sliding collar 542 having a narrow deformable opening which when force is put on the lateral sliding collar deforms around the hub or base and then locks the needle in the larger space. This can be used in association with other locking mechanisms is the internal surface of the collar with the locking mechanisms.

Also, as in the first embodiment, examples of locking devices that would function with these designs includes tapered and/or interlocking rings or tabs; oppositely directed and interlocking dentates or projections whether abrupt or tapered; interlocking rings, ridges, or shaped projections that are trapped in a space created by a tapered dentate or projection and a mating surface that accommodates ties projection, or oppositely directed dentates or projections; locking tines with or without male and female components, screw and threaded fittings including standard, incomplete, and “child proof.” These are only examples, and many other locking mechanisms are possible and anticipated between the lateral collar or collar equivalent and the base; hub, or neck of the needle or the extension of the sheath or cap.

One of the aspects of this safety needle system, is that the needle be recapped after use in order to lack it within the sheath using the safety collar. Recapping or resheathing a sharp is one of the most dangerous procedures for medical personnel, and results in a high proportion of the needle sticks to medical workers. However, experiments in our laboratory have demonstrated that sheaths with handles reduce recapping or resheath sticks from about 6% to less than 0.01%, a tremendous reduction.

Thus, sheaths with handles would be most optimal for these devices. FIGS. 28-33 demonstrates examples of locking sheaths with handles.

Referring to FIG. 28, an embodiment of the invention similar to that shown in FIG. 1 is illustrated, including the additional feature of a handle. The handle 610 for the sheath with the external locking collar is shown. Since the locking collar must move down the sheath or cap to bind the needle, the handle 610 must not impeded this longitudinal movement of the collar down the sheath. To accommodate the movement of the collar, the handle is joined to the sheath above the locking collar, and a horizontal member of the handle, provides adequate room for the sheath to move down the cap without contacting the handle 610. Alternatively, the horizontal member section of the handle could join at the base of the cap or sheath, but in this case there would have to be a slit in the locking collar to accommodate the presence of the handle in this lower position.

Referring to FIG. 29, an embodiment of the invention similar to that shown in FIG. 13 is illustrated, including the additional feature of a handle. The handle 620 is shown on a sheath with an internal locking collar. In this case, the handle 620 can join anywhere on the external sheath, but not on the exterior portion of the locking collar.

Referring to FIG. 30, an embodiment of the invention similar to that shown in FIG. 20 is illustrated, including the additional feature of a handle. The handle 630 is shown on a sheath with a lateral locking collar. In this case, the handle 630 joins the lateral extension which accommodates the track for the locking collar.

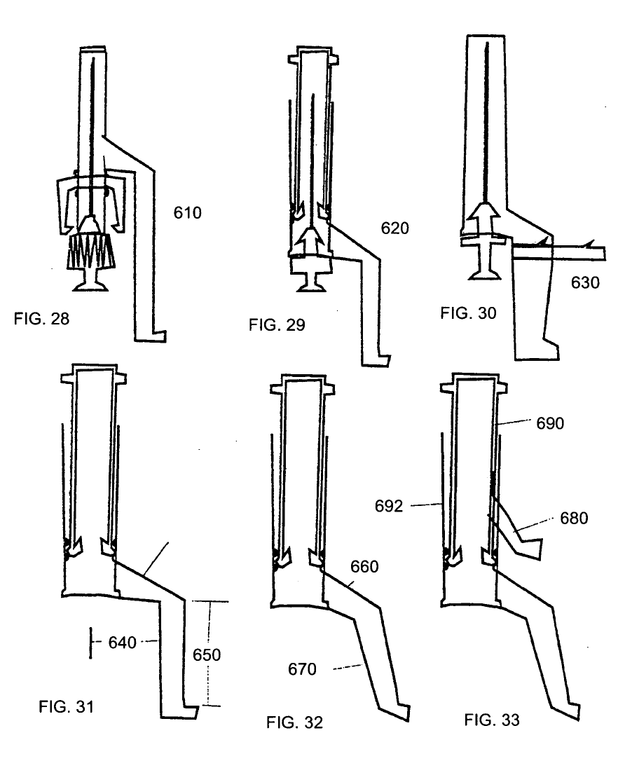

Referring to FIG. 31, a detail view of the protective cap similar to that shown in the embodiment of FIG. 29 is illustrated, showing dimensioning of the handle and demonstrates the various components of the handle. To ensure that these safety needles with locking sheath are universal, that is they will fit on any medical device, the handle must be made in a way that it does not contact or obstruct various sized medical devices. One possibility is to have the handle be horizontal (right angles to the sheath). However, that does not remove the fingers from the plane or strike zone of needle sticks. One solution, is to have a lateral (horizontal) member that puts the handle away from the centerline (center of the fitting for the needle or sharp) of the medical device a specified distance shown by the dimension 640. The inferior distance away from the needle fitting is then determined by the dimension 650 of the handle.

Referring to FIG. 32, a detail view of the protective cap similar to that shown in the embodiment of FIG. 29 is illustrated, showing an alternate handle shape. According to this alternate embodiment, the handle is placed at an angle like a angel winged that both puts the fingers laterally and inferiorly away from the strike zone. Either or both the horizontal member 660 and the handle 67j0 can be angled to accommodate medical devices that increase in size with distance from the needle fitting.

Referring to FIG. 33, a detail view of the protective cap similar to that shown in the embodiment of FIG. 29 is illustrated, showing an accessory handle connected to the internal locking collar. An accessory handle 680 can also bind to the collar through a slit or opening in the exterior sheath 692 so that the locking collar 690 can be mated to the needle without pushing on the top of the cap. This accessory handle 680 could be longer as well to take the fingers completely out of the strike zone for needle sticks. Similar handles may be embodied for the external and lateral locking collars as well.

The present invention has been described above in terms of various exemplary embodiment. It will be understood by those of ordinary skill in the art that various modifications and improvements may be made to the described embodiments without departing from the scope of the invention. Further, any reference to claim elements in the singular, for example, using the articles “a,” “an,” or “the” is not to be construed as limiting the element to the singular.

1. A safety needle system comprising:
of a needle;
a sheath sized to removably cover the needle; and
a locking collar adapted to engage the sheath to selectively and irreversibly lock the sheath over the needle.

2. The safety needle system of claim 1, wherein the locking collar is disposed internally of the sheath.

3. The safety needle system of claim 2, wherein the locking collar locks the sheath over needle by engaging both the sheath and a hub portion of the needle.

4. The safety needle system of claim 1, wherein the locking collar is disposed externally of the sheath.

5. The safety needle system of claim 4, wherein the locking collar locks the sheath over needle by engaging both the sheath and a hub portion of the needle.

6. The safety needle system of claim 1, the safety needle further comprising:
a handle extending from the sheath.
